(19) United States
(12) Patent Application Publication
(10) Pub. No.: US 2006/0211055 A1
(43) Pub. Date: Sep. 21, 2006

(54) Capture and release assay system and method

(75) Inventors: Dean G. Hafeman, Hillsborough, CA (US); H. Garrett Wada, Atherton, CA (US); Anton Simeonov, Mountain View, CA (US)

Correspondence Address: CALIPER LIFE SCIENCES, INC., 605 FAIRCHILD DRIVE, MOUNTAIN VIEW, CA, 94043-2234, US

(73) Assignee: Caliper Life Sciences, Inc., Mountain View, CA (US)

(21) Appl. No.: 11/440,615

(22) Filed: May 24, 2006

Related U.S. Application Data
(62) Division of application No. 10/705,211, filed on Nov. 7, 2003, now abandoned.
(60) Provisional application No. 60/425,582, filed on Nov. 12, 2002.

(51) Int. Cl. G01N 33/53; C12Q 1/68

(52) U.S. Cl. 435/7.5; 435/6

(57) ABSTRACT

A method for detecting one or more components of interest in a fluid-borne sample in a microchannel of a microfluidic device includes flowing the fluid-borne sample, in which the one or more components of interest are bound to a first labeled component binding moiety to form a first labeled complex, through a binding channel region of the microchannel. The binding channel region includes a second component-binding moiety reversibly bound to a wall surface of the binding channel region. At least a portion of the one or more components of interest is bound to the second component-binding moiety to form a second labeled complex. The second labeled complex is released from the binding channel region and flowed through a detection channel region of the microchannel, where the second labeled complex is detected.

### CROSS-REFERENCE TO RELATED APPLICATION

This application is a divisional of U.S. patent application Ser. No. 10/705,211 filed Nov. 7, 2003, which claims the benefit of U.S. Provisional Patent Application No. 60/425,582, filed Nov. 12, 2002, both of which are incorporated herein by reference in their entirety for all purposes.

### BACKGROUND OF THE INVENTION

Analytic detection of biomolecules, e.g., proteins, nucleic acids, and the like, is fundamental to molecular biology. In many applications, it is desirable to detect the presence of one or more particular molecules in a sample. For example, identification of a particular DNA sequence within a mixture of restriction fragments is used to determine the presence, position, and number of copies of a gene in a genome. It is also an integral technique in DNA typing. Analytic detection is also used, e.g., in disease diagnosis and drug development, to determine the presence of a particular antibody or protein, e.g., in a blood sample or large chemical library. For example, immunoassays have been developed for the detection of Alpha-fetoprotein (AFP), which is an early fetal plasma protein, the functional equivalent of albumin, which is produced by the fetal yolk sac, liver, and gastrointestinal tract.

Elevation of serum AFP to abnormally high values occurs in several malignant diseases, most notably nonseminomatous testicular cancer and primary hepatocellular carcinoma. In the case of nonseminomatous testicular cancer, a direct relationship has been observed between the incidence of elevated AFP levels and the stage of disease. Elevated AFP levels have also been observed in patients diagnosed with seminoma with nonseminomatous elements, but not in patients with pure seminoma.

In addition, elevated serum AFP concentrations have been measured in patients with other noncancerous diseases, including ataxia telangiectasia, hereditary tyrosinemia, neonatal hyperbilirubinemia, acute viral hepatitis, chronic active hepatitis, and cirrhosis. Elevated serum AFP concentrations are also observed in pregnant women.

Detection of biomolecules, such as AFP in human serum, is therefore of fundamental value in diagnostic medicine, and in other fields as well such as archaeology, anthropology, modern criminal investigation, and the like. To meet these needs many techniques, e.g., DNA blotting, RNA blotting, protein blotting, ELISA assays, and immunoassays of various forms have been developed to detect the presence of a particular molecule or fragment in the midst of a complex sample containing similar molecules. Recently, new and faster microfluidic methods of performing biological assays in microfluidic systems have been developed, such as those described by the pioneering applications of U.S. Pat. Nos. 5,942,443 and 6,391,622, the entire contents of which are incorporated by reference herein. For example, high throughput methods for analyzing biological reagents, including proteins, are described in these applications. Microfabricated glass (or quartz, plastic or silicon) substrates are rapidly becoming a convenient mechanism with which to execute liquid-phase analyses and thus a variety of techniques based on electrophoresis have been developed and implemented on microfluidic devices, including enzyme and immunoassays, DNA sequencing and PCR amplification. In these types of applications, the separated chemical analytes of interest are typically detected by a laser induced fluorescence detection method. One of the challenges of performing quantitative detection of small quantities of analytes in microchannels is the detection sensitivity of such methods. For example, in performing an immunoassay for the detection of AFP in human serum, the interference from the serum sample greatly limits the detection sensitivity for the analyte of interest.

Thus, highly sensitive and more widely applicable detection methods using laser induced fluorescence in microchannels are desirable for the detection of analytes of interest for analytical, biological, industrial, and other applications. Applicant has discovered a new and improved method for performing sensitive binding or affinity assays which take advantage of high-throughput, low cost microfluidic systems. The improved methods and systems use a “capture and release” technique for immunoconcentration of analytes in a microchannel of a microfluidic device to enhance assay sensitivity (e.g., by removing excess labeled reagent(s)) and remove serum (and other types of sample) interference during separation and detection of the analyte of interest. The present invention provides these and other features by providing high throughput microscale systems for analyte detection, affinity purification, sensitive binding assays, and the like, and many other features that will be apparent upon complete review of the following disclosure.

### SUMMARY OF THE INVENTION

The present invention provides methods, devices, and systems for capturing, releasing and detecting one or more component of interest in a sample (such as human serum), e.g., in a microfluidic system. Typically, the method involves incorporation of an affinity purification zone, or binding channel region, upstream from a separation channel region in a microfluidic device. For example, a sample containing a protein of interest, such as AFP in human serum, is flowed through an affinity purification zone or binding region, in which at least a portion of the component of interest specifically binds to a protein binding moiety, e.g., an antibody. For example, in binding assays, such as immunoassays, a binding agent such as an antibody that is complementary to an analyte to be measured, such as AFP in human serum, can be coated (using appropriate coating techniques discussed below) to a wall surface of a microchannel and used to “capture and release” the analyte for immunoconcentration of the analyte to enhance assay sensitivity and remove sample (e.g., serum) interference for subsequent detection thereof. The affinity bound protein of interest is subsequently released, in complex form bound to the component binding moiety, from the affinity purification zone and flowed through the separation region, e.g., to observe a single band corresponding to the component of interest/component binding moiety complex migrating at a characteristic mobility in the separation channel.

In one aspect, a method for detecting one or more component of interest in a microchannel of a microfluidic device is disclosed which generally comprises the steps of flowing a component binding moiety specific for the analyte of interest into a binding channel region of a microchannel of the device which region is modified (e.g., derivatized) to bind the component binding moiety to a wall surface of the region; flowing a fluid-borne sample which comprises the one or more component of interest through the binding channel region of the microchannel, which binding channel region comprises the component-binding moiety which is reversibly bound to the wall surface of the binding channel region, thereby binding at least a portion of the one or more component of interest to the component-binding moiety to form a bound complex; releasing the complex comprising the component-binding moiety and the one or more component of interest from the binding channel region thereby releasing the complex into the microchannel; and flowing the released complex through a separation channel region of the microchannel and detecting the complex. Flowing the component binding moiety and/or sample through the binding channel region optionally comprises applying pressure to the fluid in the binding channel region or electrokinetically flowing the fluid therein.

In some embodiments of the present invention, the component of interest comprises a protein (e.g., AFP) and the component-binding moiety comprises a protein-binding moiety. For example, the component-binding moiety optionally comprises an antibody specific to the component of interest. In other embodiments, the component of interest comprises a carbohydrate and the component-binding moiety comprises a carbohydrate-binding moiety, e.g., a lectin specific to a carbohydrate of interest. In addition, the component-binding moiety preferably comprises a label moiety (although in some instances the analyte of interest may also include a label). For example, the capture antibody can comprise a nucleic acid chain bound thereto which includes at least one fluorescent label associated therewith, for example, two or more fluorescent labels, to enhance the detection sensitivity of the assay.

In one particular embodiment of the present invention, the wall surface of the binding channel region of the microchannel is at least partially coated with biotin derivatized silane to which streptavidin is bound, wherein the streptavidin is used to bind a biotinylated nickel chelator that binds nickel to the wall surface of the binding channel region of the microchannel. The component binding moiety is coupled to a polyhistidine tail to mediate binding to the nickel coated wall surface of the binding channel region of the microchannel. The component binding moiety is preferably labeled, e.g., includes a nucleic acid chain bound thereto to which is associated one or more fluorescent labels. After the labeled component of interest, or at least a portion thereof, if present in the sample, is bound in the binding region to the component binding moiety, the remaining sample is typically flowed to a waste reservoir for disposal. A wash step is optionally used (e.g., a wash buffer solution is flowed through the binding channel region) to remove any residual test sample (e.g., human serum) and any unbound labeled component binding moiety to the waste reservoir. The labeled component of interest/binding moiety complex is then released, e.g., after the sample (e.g., human serum) has been sent to the waste reservoir, from the binding channel region, thereby resulting in a released complex containing the component of interest. Release of the complex from the binding channel region is optionally achieved, e.g., by adjusting the temperature or pH in the binding channel region or by, e.g., introducing one or more releasing reagent into the binding channel region, such as an appropriate elution buffer, for example, histidine, imidazole, and the like.

The released component of interest/binding moiety complex is then flowed through the separation region, e.g., electrokinetically, resulting in separation of any unbound labeled component binding moiety from the labeled released complex. The separation is typically accomplished electrophoretically in a polymer or gel disposed within the separation channel region. For example, the method typically comprises electrophoretically separating the released complex and any unbound, labeled component binding moiety in a polyacrylamide solution, matrix, or gel disposed within the separation channel region. As the complex is separated from any unbound component binding moiety, it is also typically detected and subsequently quantified. For example, the affinity bound protein can be quantitated by integrating the signal from the detector for the labeled complex and comparing the signal with known calibrators.

In a related aspect of the present invention useful for performing highly sensitive binding immunoassays, for example, the assay system utilizes at least first and second component binding moieties to perform the assay. A first unlabeled component binding moiety is bound to a wall surface in the binding channel region which has an affinity for the component of interest, as previously described. The sample in this embodiment is preferably pre-incubated with a second labeled component binding moiety to form a first labeled component binding moiety/analyte complex. The sample containing the first complex is then flowed into the binding channel region where the first complex binds to the first unlabeled component binding moiety via the analyte of interest to form a second, ternary complex (e.g., labeled capture antibody/analyte/capture antibody) resulting in the analyte (e.g., AFP molecules) being sandwiched between the unlabeled solid phase and labeled component binding moiety. The binding channel region can then be washed to remove any residual test sample and any unbound, second labeled component binding moiety which is directed into a waste reservoir on the device. Release of the ternary complex is then performed as described previously using, for example, an elution buffer such as an imidazole elution buffer, and the ternary complex is optionally separated (e.g., to separate the analyte of interest into its component fractions or forms, if necessary, as described further below in connection with an AFP assay), and then detected downstream in a detection region of the microchannel. The advantage of this sandwich-type assay technique is that labeled component binding moiety will be retained in the binding channel region only if the analyte of interest is present in the sample, thus eliminating interference from any unbound, labeled component binding moiety in the binding channel region as in the previous embodiment.

The component binding moiety/analyte complex is typically optionally detected, e.g., through optical detection using a label moiety as described above. For example, a luminescent, color, or fluorescent label moiety fixed to the component binding moiety (or the component of interest), or to a nucleic acid chain bound thereto, is optionally optically detected. Alternatively the label moiety may be an enzyme or catalyst that generates a measurable signal when combined with a substrate (or substrates) for the enzyme or catalyst. For example, the enzyme could be luciferase and the substrate luciferin and ATP. The combination of the luciferase label, luciferin and ATP creates a chemiluminescent signal proportional to the amount of the label present within a detection volume. The chemiluminescent signal is the emission of light from 500-600 nm in wavelength. The light may be detected by a light detection device such as a photodiode, a photomultiplier tube (PMT), a charge-coupled device (CCD), a CMOS photodetector, or the like. Still another label moiety could be a light-absorbing chromophore, light-reflecting or light-scattering or fluorescing particles, such as quantum dots, nanobarcodes or gold particles which may be used to create optically absorbing or Raman-scattering light signals. The detection volume is the volume of label moiety that is monitored by the light detection device employed.

In some embodiments, the method further comprises labeling the component binding moiety with a fluorescent label, e.g., in a microfluidic channel, and detecting the analyte of interest in the complex by detecting the fluorescent label, e.g., in a detection channel region.

The binding channel region, which is modified to capture the component-binding moiety, is upstream from a separation channel region. In some embodiments, the binding channel region is a derivatized channel or channel region. The binding channel region optionally comprises one or more particle set comprising a plurality of particle member types, e.g., particle member types comprising a component binding moiety. The particle member types, which are typically about 0.1 μm to about 50 μm in diameter, optionally comprise a polymeric material, a silica material, a ceramic material, a glass material, a magnetic material, a metallic material, an organic material, or the like. Example materials include, but are not limited to, polyvinylidene fluoride (PVDF), polyamide, nylon, nitrocellulose, polystyrene, PMA, glass, and the like. Binding a component binding moiety to a particle set optionally comprises adsorbing the component binding moiety onto one or more members of such a particle set. The component of interest then binds to the particle set via specific binding with the component binding moiety.

In another aspect, the present invention provides microfluidic devices and systems for detecting a component of interest in a sample. A typical microfluidic device comprises a body structure with a plurality of covered microscale channels disposed therein. As used herein, a “microscale” or “microfluidic” channel typically means that the channel will have at least one very small cross sectional dimension, e.g., in the range of from about 0.1 micron to about 500 microns. Preferably the cross-sectional dimensions of the channels will be in the range of from about 0.1 to about 200 microns and more preferably in the range of from about 0.1 to about 100 microns.

In some embodiments, the devices optionally comprise a fluid direction system fluidly coupled to the body structure and operably coupled to a control system, such as a computer, to control the flow of fluids through the device. The fluid direction system transports, e.g., the sample and other fluid-borne components, through the microscale channels as directed by the control system. Exemplary fluid direction systems comprise an electrokinetic based fluid direction system and/or a pressure based fluid direction system, and/or a hybrid electrokinetic/pressure based fluid direction system. For example, the sample is optionally flowed through the binding region by the application of pressure from a pressure based fluid direction system and flowed through the separation channel electrophoretically using an electrokinetic based fluid direction system.

The devices also optionally comprise one or more “sippers” or capillaries of microscale dimension fluidly connected to the binding channel region or other channel of the device, upstream from the binding region. One or more sources of the sample are then typically fluidly connected to the binding region, e.g., via the one or more sipper or capillary, for introducing samples into the devices. The devices will typically include a detection region downstream of the separation channel region. A detection system, e.g., a chemiluminescent, fluorescent, or calorimetric detector, is typically positioned proximal to such a detection region for detecting the labeled complex as it is eluted from the separation region. The detector will typically be operably coupled to a computer which includes software for analyzing the one or more signals produced by the detection system. The software typically comprises at least a first instruction set for deconvoluting the data and quantitating the affinity bound analyte of interest by integrating the signal from the detector for the labeled complex and comparing the signal with known calibrators.

### BRIEF DESCRIPTION OF THE FIGURES

FIG. 1: Schematic of an example channel configuration of the invention in which the binding region and the separation region comprise a single channel.

FIGS. 2A-D: Schematic of an exemplary binding channel region of the microchannel of FIG. 1 showing one capture and release assay system and method according to the teachings of the present invention.

FIG. 3: A sample plot of fluorescence versus time for an exemplary assay using the assay system and method of FIGS. 2A-D.

FIGS. 4A-D: Schematic of an exemplary binding channel region of the microchannel of FIG. 1 showing a second embodiment of a capture and release assay system and method according to the teachings of the present invention.

FIG. 5: A sample plot of fluorescence versus time for an exemplary assay using the assay system and method of FIGS. 4A-D.

### DETAILED DISCUSSION OF THE INVENTION

The present invention provides new technology for detecting a component of interest in a complex sample. The present invention provides methods and microscale devices for continuous flow binding (e.g., capture), release, separation and detection. Prior to separation, the sample is flowed through a binding channel or affinity purification zone in which it is contacted by a moiety that specifically binds the one or more component of interest within the mixture. For example the component of interest is optionally a protein that is detected by a binding reaction with an antibody specific to the protein. The component of interest is detected in the present invention by binding the component of interest to a binding moiety that is specific to the component of interest. The component of interest is optionally a protein, a peptide, a nucleic acid (e.g., an oligonucleotide, DNA, cDNA, or RNA), carbohydrate, or the like. The component is typically included in a complex mixture of various components, e.g., other proteins, nucleic acids, and the like. For example, the component of interest is optionally a component of a cell extract or human serum sample or other tissue or fluid sample, e.g., cerebrospinal fluid or urine.

In a preferred embodiment, such as in sensitive binding assays such as immunoassays, the component of interest is a protein such as AFP. A “protein,” as used herein, refers to a polymer of amino acid residues. The term also applies to amino acid polymers in which one or more amino acid residues are linked to carbohydrate moieties of polysaccharide chains forming “glycoproteins” or to lipid moieties forming lipoproteins. The term applies to amino acid polymers in which one or more amino acid residue is an analog or mimetic of a corresponding naturally occurring amino acid or in which one or more amino acid residue is a modified naturally occurring amino acid residue (e.g., modified with the addition of, e g., chemical groups such as methyl groups), as well as to naturally occurring amino acid polymers. A protein of biological or other interest is optionally detected using the methods, devices, and systems of the present invention.

As well as proteins, carbohydrates, and nucleic acids referenced above, the component of interest, or “analyte”, can include any substance susceptible to detection using the instant invention. Other exemplary analytes include chemical and biochemical moieties, such as peptide hormones, non-peptide hormones, drugs of abuse, environmental pollutants, pharmaceuticals, microbial antigens, viral antigens, carbohydrates, polyclonal antibodies, monoclonal antibodies, anti-idiotypic antibodies, antibody fragments, enzyme substrates, enzyme inhibitors, biotin, and receptors. The currently preferred analytes are those of clinical significance, such as hormones, proteins, glycoproteins, lipoproteins, peptides, microbial antigens, viral antigens, whole viral particles, microbial and viral antibodies, and vitamins. It should be further understood that biochemical or chemical substances which can be rendered amenable to complex formation, i.e., can be manipulated or modified to bind with at least one different binding moiety, are considered suitable for use in the claimed invention.

As used herein, the term “sample” is intended to mean any specimen to be analyzed for an analyte of interest. Currently preferred samples include, but are not limited to, any biological or environmental specimen suspected to contain an analyte of interest. Samples suitable for use in the claimed invention can include body fluids of human and animal origin including, but not limited to: blood, serum, plasma, urine, cerebrospinal fluid, saliva, sweat, semen, vaginal fluid, amniotic fluid, and ascites fluid. Samples of plant or microbial origin such as cellular, spore, plasmid, or viral extracts are also suitable for use in the claimed invention. Additionally, samples may include fluids such as, but not limited to, rain water, ocean water, ground water, soil extracts, atmospheric aerosol, and sewer water which may be analyzed for environmental pollutants or toxins.

As used herein, the term “component binding moiety” is intended to mean any moiety capable of binding activity. The term component binding moiety includes, but is not limited to, any biochemical or chemical moiety which has an ability to interact specifically with, and bind with, a corresponding component of interest or analyte. It will be obvious to those skilled in the art that, in order to practice the claimed invention, the identity of the particular binding moiety will be governed by the identity of the particular analyte to be detected. Generally speaking, binding moieties suitable for use in the instant invention include, but are not limited to, the following biochemical and chemical moieties: proteins, peptides, nucleic acids, peptide hormones, non-peptide hormones, environmental pollutants, lectins, microbial antigens, viral antigens, carbohydrates, polyclonal antibodies, monoclonal antibodies, anti-idiotypic antibodies, antibody fragments, biosynthetic antibody binding sites, DNA binding proteins, signal transduction proteins or moieties such as protein phosphorylation sequences, enzymes and modulated enzymes such as activated enzymes from proenzymes, avidin, and receptors. Moreover, biochemical or chemical substances which can be rendered amenable to complex formation with a corresponding analyte, i.e., can be manipulated or modified to bind with a particular analyte, are considered suitable for use in the instant invention. Currently preferred binding moieties are those suitable for detection of clinically significant analytes. Identification of other binding moieties and equivalents thereof is well within the skill of the ordinary practitioner and would require no more than routine experimentation.

In one preferred embodiment, the binding moiety preferably comprises at least one charge modifying moiety and at least one detectable label to facilitate separation of the analyte/binding moiety complex in the separation region of the microchannel and subsequent detection thereof. For example, the binding moiety can include a nucleic acid chain which is modified to include at least one label, e.g., two or more labels, e.g., four or more labels, to improve the sensitivity of detection of the analyte of interest. For example, in one non-limiting embodiment of the invention, DNA containing multiple fluorescent labels is used as the component binding moiety. Incorporation of a predetermined number of fluorescent labels at defined positions within the DNA can be accomplished, for example, by the used of multiply labeled polymerase chain reaction (PCR) primers. Those primers, in turn, can be prepared by the following sequence of synthetic steps. First, an oligonucleotide is synthesized which incorporates amino-dT instead of the regular T-base at predetermined positions within the oligonucleotide which are chosen such that subsequent close interaction and intramolecular dye aggregation is minimized. One can optionally synthesize the oligonucleotide with a 5′-amine as well, in order to place one dye label on the 5′ end of the molecule. The oligonucleotide is then reacted with an activated dye of choice (e.g., AlexaFluor 647 carboxylic acid succinimidyl ester, commercially available from Molecular Probes, Eugene, Oreg.), which results in the dye being covalently attached to one or more of the amino-dT modified bases. A separation method of choice (e.g., strong anion exchange HPLC or other standard separation technique known to those of ordinary skill in the art) can then be used to separate and isolate the population of oligonucleotide molecules carrying the highest number of labels and use the resulting compound(s) as PCR primer(s). Alternatively, PCR can be performed using conventional primer preparation, followed by isolation of the DNA product containing a desired number of labels. Nonlimiting examples of other appropriate binding moiety conjugates are described in detail in PCT Patent Application No. WO 02/082083, which is incorporated by reference in its entirety herein.

A charge-modifying moiety is any moiety which imparts to the component binding moiety the ability to electroseparate analyte-containing complex from unbound binding moiety such that the separation or shift in mobility is sufficient to permit detection of the complex. Any such charge-modifying moiety is suitable for use in the instant invention. A charge-modifying moiety suitable for use in the instant invention can impart a negative charge or a positive charge. The skilled artisan would be able to determine the identity of a suitable moiety by routine experimentation. Nonlimiting examples of charge modifiers suitable for use in the present invention include the following moieties: oligonucleotides (synthetic DNA), cDNA molecules (e.g., single stranded or double stranded DNA), RNA, polyaspartic acid, polyglutamic acid, polysulfonated tyrosine, carboxymethylcellulose, polymaleic acid, polylactic acid, polysulfonic acid, and polyacrylic acid. Other suitable charge modifiers include: polylysine, polyethyleneimines, polyarginine, and poly(diallyl dimethyl ammonium) salts. Currently, insofar as negative charge modifiers are concerned, polyT can be used; polyA, polyC, or polyG can equally well be used. Other charge modifiers are polymers containing (—OH) groups such as polysaccharides or polydiols. Alternatively, charged polymers may be any polymer with attached charge groups such as sialic acid groups, phosphate groups, sulphonate groups, carboxylic acid groups, amine groups, and the like. Such groups will form complexes with borate if borate is included in the separation medium and impart a negative charge. Generally speaking, any charge-modifying moiety having approximately a range of 3 to 500 negative charges or 3 to 500 positive charges is suitable for use in the instant invention, however, the skilled artisan will be able to identify the charge best suited to his/her particular application using only routine experimentation.

Detectable moieties or labels, as used herein, are moieties suitable for use in the claimed invention including, but not limited to: enzymes, fluorophores, chromophores, radioisotopes, electrochemical moieties, and chemiluminescent moieties. A currently preferred detectable moiety is a fluorescent moiety, for example rhodamine or Alexa dye. Other currently preferred detectable moieties include: fluorescein, cyanine dyes (e.g., Cy-3, Cy-5), coumarins, phycoerythrin, phycobiliproteins, dansyl chloride, and Texas Red.

In one embodiment, a component-binding moiety of the present invention is optionally a “protein-binding moiety” specific to a protein of interest. The protein-binding moiety is any molecule, e.g., a protein (e.g., AFP), a nucleic acid, an antibody, an enzyme, or the like, that specifically binds to a protein of interest in the present invention. The phrase “specifically binds” to a protein or component refers to a binding reaction that is determinative of the presence of the protein in a heterogeneous population of proteins, components, and other biologics. Thus when a protein-binding moiety specific to a protein of interest binds to that protein (or when a component-binding moiety specific to a component of interest binds to the component of interest) it binds to that particular protein or component preferentially out of a complex mixture. For example, it binds at least two times the background, more typically 10 to 100 times background, and does not substantially bind in significant amounts to other proteins or components in the sample. Specific binding to a polyclonal antibody may require an antibody that is selected for its specificity for a particular protein or component as discussed below.

In one typical embodiment, the “component-binding moiety” is a “protein-binding moiety,” such as an antibody, receptor, or ligand. An “antibody” is a multifunctional glycoprotein produced in nature by an immune system. Antibodies function in the immune system to prevent infection by microorganisms. They perform this function by recognizing and binding to particular molecular configurations on invading microorganisms, each antibody being able to bind only one or a small number of related molecular configurations or antigens. Typically, an antibody comprises a framework from an immunoglobulin gene or fragment that specifically binds and recognizes the antigen. The recognized immunoglobulin genes include the kappa, lambda, alpha, gamma, delta, epsilon, and mu constant region genes as well as the various immunoglobulin variable genes. Light chains are classified as either kappa or lambda. Heavy chains are classified as gamma, mu, alpha, delta, or epsilon, which define the immunoglobulin classes, IgG, IgM, IgA, IgD, and IgE.

An exemplary immunoglobulin or antibody structural unit comprises a tetramer. Each tetramer is composed of two identical pairs of polypeptide chains, each pair having one light and one heavy chain. The N-terminus of each chain defines a variable region of about 100-110 or more amino acids primarily responsible for antigen recognition.

Antibodies exist as intact immunoglobulins or as a number of well-characterized fragments produced by digestion with various peptidases. While various antibody fragments are defined in terms of the digestion of an intact antibody, one of skill will recognize that such fragments may be synthesized de novo either chemically or by using recombinant DNA technology.

For preparation of monoclonal or polyclonal antibodies, any technique known in the art is optionally used. (see, e.g., Paul (ed.) (1993) Fundamental Immunology, Third Edition Raven Press, Ltd., New York Coligan (1991) Current Protocols in Immunology Wiley/Greene, NY; Harlow and Lane (1989) Antibodies: A Laboratory Manual Cold Spring Harbor Press, NY; Stites et al. (eds.) Basic and Clinical Immunology (4th ed.) Lange Medical Publications, Los Altos, Calif., and references cited therein; Goding (1986) Monoclonal Antibodies: Principles and Practice (2d ed.) Academic Press, New York, N.Y.; and Kohler and Milstein (1975) Nature 256:495-497. Other suitable techniques for antibody preparation include selection of libraries of recombinant antibodies in phage or similar vectors. See, Huse et al. (1989) Science 246:1275-1281; and Ward et al. (1989) Nature 341:544-546. Specific monoclonal and polyclonal antibodies and antisera will usually bind with a Kd of at least about 0.1 μM, preferably at least about 0.01 μM or better, and most typically and preferably, 0.001 μM or better.

In other embodiments, the component-binding moiety comprises biotin, avidin, lectin, a small organic molecule, a specific DNA sequence, a specific RNA sequence, or the like. For example, when the component of interest is a carbohydrate, the component-binding moiety is preferably a lectin, e.g., a glycoprotein that binds oligosaccharides or carbohydrates typically through precise and/or stereospecific interactions. When the component binding moiety comprises a specific DNA sequence or RNA sequence, the analyte can be conjugated (using techniques well known to those of ordinary skill in the art) to an antisense DNA or RNA molecule specific to the respective DNA sequence or RNA sequence, for example. Another binding system of interest in the present invention is the avidin-biotin system. Biotin is optionally linked to proteins or nucleic acids and used as a label. Detection of a biotinylated protein or nucleic acid may occur due to the enzymatic or chemiluminescent reaction of biotin with a detector complex comprising streptavidin or avidin, which binds tightly to the biotin.

In one preferred embodiment of the invention, the component binding moiety is preferably reversibly coated to the inner wall surface of a microchannel using a variety of techniques. For example, in one non-limiting example of a binding assay according to the teachings of the present invention, the wall surface of a microchannel is coated with biotin derivatized silane to which streptavidin is bound. The streptavidin is then used to bind a biotinylated nickel chelator (e.g., nitrilotriacetic acid (“NTA”)) that binds nickel (other appropriate metal cation, such as cobalt, e.g., cobalt-based TALON™ sold by BD Biosciences Clontech, Palo Alto, Calif.) to the wall of the binding region of the channel. The nickel is then ready to capture a 6×His-tagged protein or capture antibody specific to the analyte of interest. For example, the binding moiety can include a capture antibody specific for the analyte of interest which is coupled to a polyhistidine tail, either by chemical coupling or genetic engineering techniques, to mediate binding to the nickel coated glass (or silica or quartz or polymeric) wall surface. The polyhistidine-tagged capture antibody binds reversibly to the nickel chelated by NTA on the wall surface of the microchannel in the binding channel region and can be easily released therefrom by flowing an appropriate elution buffer through the system. As described previously, the capture antibody can include a nucleic acid marker having at least one label associated therewith to facilitate separation and detection of the analyte/antibody complex in the separation and/or detection channel regions of the microchannel.

The sample containing the analyte of interest is flowed into the binding channel region where the analyte binds to the wall of the channel via the capture antibody. For some period of time, as the sample flows through the channel, the analyte is affinity concentrated in the channel, as much as 5×, 10×, 100×, 1000×, or more. A short wash step using a buffered solution can be used for the concentrated analyte if necessary to remove interfering substances (e.g., human serum) from the analyte of interest. After being bound in the binding channel region by the capture antibody, the analyte/antibody “complex” can be released from the binding channel region, e.g. eluted therefrom, by flowing an appropriate buffer, e.g., imidazole buffer, through the system. The analyte/antibody complex can then be separated from excess released (unbound) labeled binding moiety, and detected after being released from the binding channel region. Other high salt solutions could optionally be used to perform the elution step such as use of the zwitterion betaine. The use of a zwitterion elution buffer has the advantage of not substantially interfering with the subsequent electrophoretic separation step because of its low electrophoretic conductivity.

Other systems could also be used to reversibly bind the component binding moiety (e.g., capture antibody) to the inner wall surface of the microchannel. For example, the binding moiety can include a disulfide linker such as SPDP (3-(2-Pyridyldithiopropionic Acid N-hydroxy-succinimide ester)) that can be reduced using dithiothreitol to release the bound analyte/antibody complex. Additionally, reversible ionic interaction could be used as the capture step. For instance, the capture antibody could be coupled to a highly charged molecule, such as DNA, RNA or other charged polymers, that dramatically increases its affinity to the wall surface of the microchannel in the binding channel region or to an ionizable coating (bearing a net charge opposite to the highly charged molecule to be captured), e.g., 3-aminopropylsilane on the channel. A high salt concentration solution, such as a Zwittergen or betaine elution buffer, could then be used to elute the analyte/antibody complex from the channel wall to transfer the complex into the separation channel of the chip for subsequent separation and/or detection thereof. In addition, the binding moiety can include a photo-cleavable linker such as a 1-2-(nitrophenyl)-ethyl moiety that can be cleaved from the coated channel surface (e.g., a streptavidin coated solid surface) using light in the wavelength range of about 300 to 360 nm. Other photo-cleavable linkers known to those of ordinary skill in the art, such as 2-nitrobenzyl, 5′-biotin phosphoramidites, amino-modifier phosphoramidites, and spacer and linker phosphoramidites, can also be used in practicing the methods of the instant invention (see, e.g., Link Technologies, Technical Summary of Photo-Cleavable Modification Reagents, Technical Information Sheet TIS-PC-01 v. 2.0, which can be found at http://www.linktech.co.uk/Downloads/TIS-PC-01%20v%202.0.pdf, the entire contents of which are incorporated by reference herein).

In addition, in other embodiments, other systems can be used to facilitate separation of the analyte of interest from the component binding moiety itself so that an appropriately labeled analyte of interest can be detected unbound from the analyte/antibody complex formed in the binding channel region. For example, for the avidin-biotin system described above, modified avidin (e.g., capped avidin) could be used to reduce the affinity of biotin for avidin and thus make the binding of the analyte to the capture antibody reversible. Although binding of biotin to native avidin or streptavidin is essentially irreversible, appropriately modified avidins can bind biotinylated binding moieties reversibly, making them valuable reagents for isolating and purifying biotinylated analytes of interest from complex mixtures (such as human serum). For example, selective nitration of tyrosine residues in the four biotin-binding sites of avidin considerably reduces the affinity of the protein for biotinylated molecules above pH 9. Consequently, biotinylated binding moieties can be adsorbed at neutral pH or below and released at ˜pH 10. Various forms of CaptAvidin agarose available commercially from Molecular Probes (Eugene, Oreg.) are particularly useful for separating and purifying biotin conjugates from complex sample mixtures.

Additionally, modified biotin can be used to provide a means of labeling and separating biomolecules, including live cells, under extremely gentle conditions. For example, DSB-X biotin reagents commercially available from Molecular Probes, which are derivatives of desthiobiotin with an additional seven-atom ‘X’ spacer, have moderate affinity for avidin and streptavidin that is rapidly reversed by low concentrations of free biotin or desthiobiotin at neutral pH and room temperature. This technique permits capture and release of DSB-X biotin-labeled molecules using a DSB-X Biotin Bioconjugate Isolation Kit or Captivate ferrofluid streptavidin also available from Molecular Probes.

The sample containing the analyte of interest can either be pre-incubated with a labeled component binding moiety to form a first binding moiety/analyte complex, or the labeled binding moiety can be first flowed into the binding channel region and adsorbed to a wall surface therein for subsequent binding to the analyte of interest. In the former assay method, the complex formed by reacting the labeled, charged binding moiety with the analyte of interest is flowed through the binding channel region which contains a second, unlabeled binding moiety that reacts again with the labeled, charged binding moiety or directly with the analyte of interest in such a way that the complex of analyte and labeled, charged binding moiety is bound in the channel and separated from the mixture of components present in the sample. The complex is thereby “captured” and concentrated in the binding region of the channel by passing multiple channel volumes of complex through the binding region. A brief wash with buffer through the binding region of the channel would complete the separation of bound complex from the mixture. The binding of the complex in the binding region is a reversible binding reaction such that by altering the buffer conditions, temperature, or by adding a dissociating agent the bound complex is released for analysis in the separation channel. The electrophoretic mobility and detectable signal from the complex of charged, labeled binding moiety and analyte of interest is used to identify and quantitate the analyte of interest. The electrophoretic mobility is used to improve the specificity of the assay by distinguishing analytes with similar binding properties but different charge and size properties. In this case, multiplexed assays can be run for two or more related or unrelated analytes that can be run simultaneously in the same separation channel by designing the charged, labeled binding moiety or mixture of binding moieties to give the different complexes different mobilities. Alternatively, the assay can be multiplexed by using two or more component binding moieties each labeled with a different fluorophore to color segregate the different formed complexes.

I. Microfluidic Systems

The microfluidic devices of the present invention are used to detect the presence of a particular component of interest, e.g., a protein of interest. The devices generally comprise a body structure with a plurality of covered microscale channels fabricated therein. For example, the present system comprises one or more of: a separation channel or channel region, a binding channel or channel region or an affinity purification zone, a waste channel for un-reacted sample components, a detection region, and the like. The channels are fluidly coupled to each other and to various reservoirs or other sources of materials. In addition, a device of the invention typically includes a control system operably coupled to the body structure for directing the flow of sample materials through the plurality of channels. Materials used in the present invention include, but are not limited to, buffers, e.g., separation buffers, binding buffers, or elution buffers, washing solutions, dyes, one or more samples, a particle set, e.g., comprising a component binding moiety such as an antibody, and the like.

For example, the control system typically directs the flow of a fluid-borne sample through a binding region and then into a separation region. In the binding region, a component of interest (either unbound or bound to a labeled component binding moiety) in the sample is bound to a component-binding moiety. The remaining components in the sample are directed to a waste reservoir for disposal. The control system also directs the bound component of interest/binding moiety complex, upon release, to flow through the separation channel region and detection region for detection thereof.

The devices of the present invention typically include a separation channel. Preferably, the separation channel is a gel filled channel, e.g., a linear polyacrylamide gel filled channel or a polymer solution filled channel, e.g., a polyacrylamide polymer solution, that separates various components based, e.g., on molecular weight, wherein each component is eluted from the separation channel with a different retention time. In other embodiments, a separation channel or channel region comprises a separation buffer, e.g., that is flowed into the separation channel region, e.g., from a buffer reservoir.

A binding channel or channel region or an affinity purification zone is typically included in the microfluidic devices and systems in the present invention. The “binding channel,” “binding region,” “binding channel region,” or “affinity purification zone” is typically upstream from the separation channel. The terms “upstream” and “downstream” refer to the relative positioning of the element so described when considered in the context of the direction of flow of the material of interest during operation of the system being described. “Upstream” refers to a location in a channel or system of channels that is farther along the channel or plurality of channels in a direction that is opposite the flow of fluid or material flow, relative to a selected site or region. The term “downstream” refers to a location in a channel or microfluidic device that is farther along the channel or plurality of channels in a selected direction of fluid or material flow, relative to a selected site or region. Typically, the phrase upstream refers to the direction of flow toward a sample or buffer reservoir or source connected to a particular channel, while downstream typically refers to the direction toward the waste reservoir connected to a particular channel.

For example, the separation region is farther along in the direction of flow in the channel system than the binding region. Therefore, materials typically flow through the binding region or channel first and then into the separation channel. A component of interest, or a portion thereof, binds to a component-binding moiety in the binding region and remains there as the remaining unbound components of the mixture are flowed into a waste reservoir.

In some embodiments, the binding channel or region comprises a derivatized channel or channel region. For example, the channel is derivatized with an antibody which binds to a component of interest. The antibody is attached to or associated with the walls of the binding channel or region. Furthermore, myriad different binding moieties can be incorporated into derivatized binding channel areas, depending upon, e.g., the specific component(s) of interest to be bound in the binding region, the surface and/or lining of the channel which comprises the binding channel area, etc. Additionally, levels of derivatization are optionally adjusted to produce the proper density of binding moieties in the binding channel areas for particular assays, etc.

Derivatization of the channel surfaces of binding channel areas optionally includes multiple rounds of additions of, e.g., derivatizing agents, functional groups comprising binding moieties for the component(s) of interest, appropriate buffers, etc. In typical embodiments, one or more rounds of one or more derivatizing agents are contacted to the substrate surface of the microchannel which comprises the binding channel. Such derivatizing agents change/modify the surface of the substrate in the binding region (i.e., they derivatize it) either in preparation for additional steps (such as addition of functional binding moieties) or as a final step (i.e., the derivatization produces the proper binding moieties in the binding region). Alternatively, the round(s) of derivatizing agents are followed by, e.g., one or more rounds of one or more agent which comprises the binding moiety for the component of interest and which binds with and/or interacts with the derivatized surface of the channel comprising the binding region.

Processes of construction of derivatized surfaces capable of incorporation into the devices and methods of the current invention are well known by those in the art. For example, see, e.g., U.S. Pat. Nos. 5,885,470 and 5,919,523, both of which are incorporated herein for all purposes.

In other embodiments, the binding channel region comprises one or more particle set, e.g., for binding the component of interest. The particle set in this embodiment typically comprises a plurality of member types comprising one or more of a silica material, a ceramic material, a glass material, a magnetic material, a metallic material, an organic material, or the like. The particle member types typically comprise a component binding moiety to which a component of interest binds, e.g., as it is flowed through the binding region. For example, the component-binding moiety is optionally adsorbed onto one or more member of the particle set. Such particles or beads typically comprise a material such as PVDF, polyamide, nylon, nitrocellulose, or the like. The component of interest typically binds to the component-binding moiety which is adsorbed onto the particles or beads. In other embodiments, the component of interest adsorbs directly onto the particles or beads, where it remains until released. Particles or beads of use in the present invention are typically about 0.1 μm to about 50 μm in diameter and are described in more detail in U.S. Ser. No. 09/510,626 filed Feb. 22, 2000, entitled “Manipulation of Microparticles In Microfluidic Systems,” by Mehta et al.

The binding region or affinity purification zone also optionally comprises a stacking region. A stacking region provides a particle retention or capture region for fixing in place a particle set, which is optionally fixed in place or mobile. The particle retention region or stacking region optionally includes a region of increased or decreased microchannel depth or width or other physical barrier (e.g., a groove, mesh, net, matrix, etc.), an electromagnetic field or porous matrix (e.g., sieving matrices), or other means of inhibiting particle movement in, or adjacent to, the stacking region. For more discussion of particle retention regions, see, U.S. Ser. No. 09/510,626 filed Feb. 22, 2000, entitled “Manipulation of Microparticles In Microfluidic Systems,” by Mehta et al.

Other features of the present devices are also optionally included, e.g., upstream or downstream from the binding channel and/or separation channel. For example, a reservoir for a binding buffer or a sample well is optionally upstream from the binding channel or channel region. Reservoirs are locations or wells, e.g., disposed within the device, at which samples, components, reagents, and the like are added into the device for assays to take place. Introduction of these elements into the system is carried out as described below. The reservoirs are typically placed so that a sample or reagent is added into the system upstream from the location at which it is used. For example, wash and elution buffer solutions are optionally added into the binding channel region from a reservoir fluidly coupled to a channel upstream from the binding channel region.

Sipper capillaries are also optionally used to introduce samples into channels of the present devices. A typical sipper capillary is fluidly coupled to a source of a plurality of samples, e.g., a microwell plate, and to a channel disposed within the device. The sipper is used to introduce a sample from the microwell plate into the microfluidic channel system. In the present devices, a sipper capillary would typically be coupled to a channel or channel region upstream from the binding region to introduce samples into the binding regions.

Another feature optionally present in the devices of the invention is a heating zone. A heating zone is typically a channel or channel region in which components or samples are heated, e.g., prior to separation. For example, a heating zone is typically positioned upstream from a separation region or channel to provide for heating of components prior to separation, e.g., by electrophoresis. Heating zones typically comprise a temperature control element operably coupled to the heating zone, e.g., for heating a mixture of components or a released component of interest, e.g., prior to flowing such components through the separation channel or channel regions. Various channel configurations and/or sizes are optionally used to provide heating zones. Variations in channel thickness and/or voltage applied to a channel are optionally used to selectively heat a particular channel region, e.g., a heating zone. See, e.g., U.S. Pat. No. 6,174,675. For example, joule heating, as provided in U.S. Pat. No. 6,306,590, is optionally used.

Detection regions are also included in the present devices. The detection region is optionally a subunit of a channel, or it optionally comprises a distinct channel, that is fluidly coupled to the plurality of channels in the microfluidic device. The detection region is optionally located at the elution point of the separation channel or region or downstream of the elution point. For example, a detection region located at the most downstream point or end of the separation channel detects separated components as they are eluted from the separation region or channel. The detection region is optionally located at the downstream end of the device just upstream from a waste well. A detection region is optionally located at whatever point in the device that detection of the components is desired. Furthermore, multiple detection regions are optionally present in various embodiments of the present invention.

The detection window or region at which a signal is monitored typically includes a transparent cover allowing visual or optical observation and detection of the assay results, e.g., observation of a calorimetric or fluorometric signal or label. Examples of suitable detectors are well known to those of skill in the art and are discussed in more detail below.

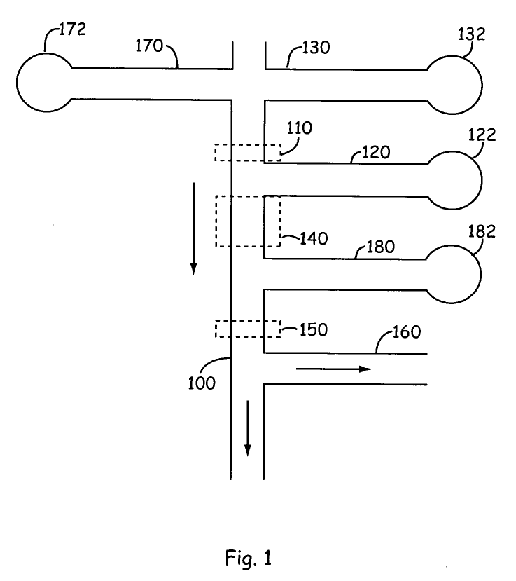

One embodiment of a channel configuration for use in a microfluidic device used in the present system is illustrated in FIG. 1. A sample, e.g., a mixture of components present in human serum, is typically introduced into channel 100, e.g., from a sipper capillary (not shown) fluidly coupled to channel 100 and a microwell plate comprising a plurality of samples, or from one or more sample reservoirs on the microfluidic device fluidly coupled to channel 100. For example, the source of a sample, wash buffer, elution buffer and the like can be a microwell plate external to the body structure of the device, having, e.g., at least one well with the selected sample or buffer solution. Alternatively, the source of a sample or other fluid can comprise a well disposed on the surface of the body structure, a reservoir disposed within the body structure, a container external to the body structure comprising at least one storage compartment, or a solid phase structure comprising the selected component or reagent in lyophilized or otherwise dried form.

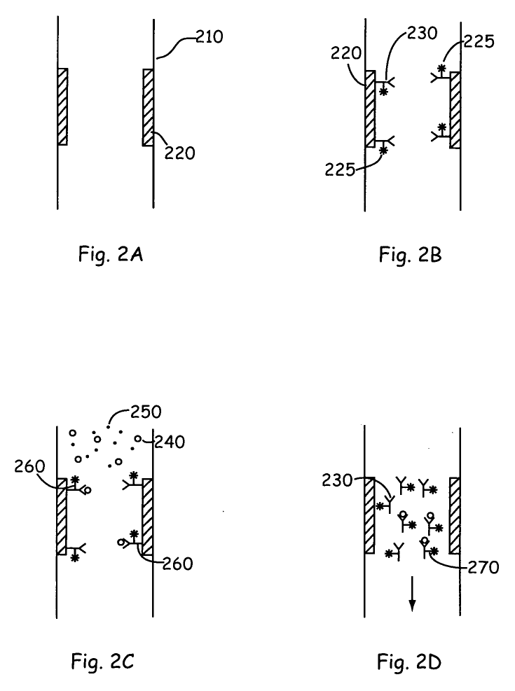

The sample is typically directed into binding channel region 110 (typically via pressure as explained herein). A binding buffer is also optionally added, which binding buffer is optimized to facilitate attachment of a component of interest to a component binding moiety as will be explained in greater detail below with reference to FIGS. 2A-D. A component-binding moiety is optionally pre-disposed within binding region 110 or added to binding region 110 from an external or internal source, e.g., a reservoir in the microfluidic device, whenever a sample is flowed through binding region 110. In binding region 110, the sample is mixed with the component-binding moiety, e.g., which is optionally attached to a particle set, which is optionally added into binding region 110 from a particle well or pre-disposed in binding region 110, e.g., in a stacking region located therein. The component binding moiety captures or binds the component of interest or a portion thereof from the mixture of components to form a bound complex containing the component of interest and binding moiety. The bound complex then remains in binding region 110 as the remaining mixture of components present in the sample is directed into channel 120 and flowed into waste reservoir 122 for disposal (or retrieval if appropriate). The complex in binding region 110 is then typically released from the binding region 110, e.g., by introducing an elution buffer solution from well 132 which solution is flowed into channel 100 and binding region 110 via connecting channel 130. The released complex is then directed into separation region 140 for separation by electrophoresis, and detection region 150 for detection of the released complex.

In addition, various additional reservoirs are optionally fluidly connected to channel 100 for storage and/or delivery of materials to detection region 150, separation region 140, and/or binding region 110, e.g., for delivery of, e.g., washing solutions, elution solutions, antibodies, diluents, and the like. For example, one or more side channels (e.g., channel 170) and reservoirs (e.g., reservoir 172) are optionally included in the device to introduce buffers, washing solutions, diluents, and the like, into channel 100. When the assay and detection are complete, the sample components are optionally directed to a waste well for disposal, or can be directed to a side channel (e.g., channel 160) for subsequent analysis and/or retrieval. Both more complexity (i.e., more channels, more binding regions, etc.) and more simplicity (i.e., only one main channel, only one main binding region, etc.) are optional embodiments of the current invention.

A variety of microscale systems are optionally adapted to the present invention by incorporating separations gels, particle sets, antibodies, wash solutions, dyes, diluents, elution buffers, and the like into the devices as described above. Microfluidic devices which can be adapted to the present invention by the addition of assay components are described in various issued U.S. patents by the inventors and their coworkers, including U.S. Pat. Nos. 5,699,157, 5,779,868, 5,800,690, 5,842,787, and 5,852,495, the contents of which are incorporated by reference herein.

Flowing and direction of fluids within the microscale fluidic devices may be carried out by a variety of methods. In general, fluids are flowed in a microscale system by electrokinetic (including either electroosmotic or electrophoretic) techniques, or using pressure-based flow mechanisms, or combinations thereof. In the present system, the control systems used to direct fluid flow typically include a combination of electrokinetic transport and pressure-based transport. For example, pressure is optionally used to flow samples through a binding channel and electrokinetic transport is used to inject samples into and flow samples through a separation channel in the present devices.

For example, the devices may include integrated microfluidic structures, such as micropumps and microvalves, or external elements, e.g., pumps and switching valves, for the pumping and direction of the various fluids through the device. Examples of microfluidic structures are described in, e.g., U.S. Pat. Nos. 5,271,724, 5,277,556, 5,171,132, and 5,375,979. See also, Published U.K. Patent Application No. 2 248 891 and Published European Patent Application No. 568 902.

Although microfabricated fluid pumping and valving systems may be readily employed in the devices of the invention, the cost and complexity associated with their manufacture and operation can generally prohibit their use in mass-produced disposable devices as are envisioned by the present invention. For that reason, in particularly preferred aspects, the devices of the invention will typically include an electroosmotic fluid direction system. Such fluid direction systems combine the elegance of a fluid direction system devoid of moving parts, with an ease of manufacturing, fluid control and disposability. Examples of particularly preferred electroosmotic fluid direction systems include, e.g., those described in U.S. Pat. Nos. 6,001,229 and 6,010,607, each of which is incorporated herein by reference in its entirety for all purposes.

In brief, these fluidic control systems typically include electrodes disposed within the reservoirs that are placed in fluid connection with the plurality of intersecting channels fabricated into the surface of the substrate. The materials stored in the reservoirs are transported through the channel system delivering appropriate volumes of the various materials to one or more regions on the substrate in order to carry out a desired screening assay.

Fluid transport and direction is accomplished through electroosmosis or electrokinesis. In brief, when an appropriate fluid solvent is placed in a channel or other fluid conduit having functional groups present at the surface, those groups can ionize. For example, where the surface of the channel includes hydroxyl functional groups at the surface, protons can leave the surface of the channel and enter the fluid. Under such conditions, the surface will possess a net negative charge, whereas the fluid will possess an excess of protons or positively charged ions (cations), particularly localized near the interface between the channel surface and the fluid. By applying an electric field along the length of the channel, cations will flow toward the negative electrode. Movement of the positively charged species in the fluid pulls the solvent with them causing fluid transport.

To provide appropriate electric fields, the system generally includes a voltage controller that is capable of applying selectable voltage levels, simultaneously, to each of the reservoirs, including ground. Such a voltage controller can be implemented using multiple voltage dividers and multiple relays to obtain the selectable voltage levels. Alternatively, multiple, independent voltage sources may be used. The voltage controller is electrically connected to each of the reservoirs via an electrode positioned or fabricated within each of the plurality of reservoirs.

Incorporating this electroosmotic fluid direction system into the device shown in FIG. 1 involves incorporation of an electrode within each of the reservoirs 122, 132, and 172, for example, and at the terminus of main sample channel 100 or at the terminus of any fluid channels connected thereto, whereby the electrode is in electrical contact with the fluid disposed in the respective reservoir or channel. Substrate materials are also selected to produce channels having a desired surface charge. In the case of glass substrates, the etched channels will possess a net negative charge resulting from the ionized hydroxyls naturally present at the surface. Alternatively, surface modifications may be employed to provide an appropriate surface charge, e.g., coatings, derivatization, e.g., silanation, or impregnation of the surface to provide appropriately charged groups on the surface. Examples of such treatments are described in, e.g., U.S. Pat. No. 5,885,470, which is hereby incorporated herein by reference in its entirety for all purposes.

Modulating voltages are then concomitantly applied to the various reservoirs to affect a desired fluid flow characteristic, e.g., flow of sample toward the waste reservoir 122. Particularly, modulation of the voltages applied at the various reservoirs can move and direct fluid flow through the interconnected channel structure of the device in a controlled manner to effect the fluid flow for the desired assay and apparatus.

Other methods of fluid transport are also available for situations in which electrokinetic methods are not desirable. For example, sample introduction and reaction are optionally carried out in a pressure-based system and high throughput systems typically use pressure induced sample introduction. In addition, cells are desirably flowed using pressure-based flow mechanisms.

Pressure based flow is also desirable in systems in which electrokinetic transport is also used. For example, pressure based flow is optionally used for introducing and reacting reagents in a system in which the products are electrophoretically separated. In the present system, a combination of pressure based flow and electrokinetic based flow is typically used. For example, a pressure based control system is typically used to introduce samples into the binding channel region 110 in which a component of interest is bound to a component-binding moiety, and electrokinetic based flow is typically used to separate the released complex from any unbound component binding moiety present in the binding channel region and released into the separation channel region 140.

Pressure is optionally applied to microscale elements to achieve fluid movement using any of a variety of techniques. Fluid flow (and flow of materials suspended or solubilized within the fluid, including cells or other particles) is optionally regulated by pressure based mechanisms or pressure based fluid control elements, e.g., as part of a fluid direction or control system, such as those based upon fluid displacement, e.g., using a piston, pressure diaphragm, vacuum pump, probe, or the like to displace liquid and raise or lower the pressure at a site in the microfluidic system. The pressure is optionally pneumatic, e.g., a pressurized gas, or uses hydraulic forces, e.g., pressurized liquid, or alternatively, uses a positive displacement mechanism, e.g., a plunger fitted into a material reservoir, for forcing material through a channel or other conduit, or is a combination of such forces.

In some embodiments, a vacuum source is applied to a reservoir or well at one end of a channel to draw the suspension through the channel. For example, a vacuum source is optionally placed at a reservoir located downstream from the detection region 150 in the present devices for drawing fluid into a channel, e.g., a vacuum source at a reservoir positioned at the downstream end of channel 100 in FIG. 1 applies a pressure to channel 100, thus drawing fluid, e.g., from a reservoir, sipper capillary, or the like, through channel 100, e.g., through binding region 110.

Pressure or vacuum sources are optionally supplied external to the device or system, e.g., external vacuum or pressure pumps sealably fitted to the inlet or outlet of the channel, or they are internal to the device, e.g., microfabricated pumps integrated into the device and operably linked to the channel. Examples of microfabricated pumps have been widely described in the art. See, e.g., published International Application No. WO 97/02357.

Another alternative to electrokinetic transport is an electroosmotic pump which uses electroosmotic forces to generate pressure based flow. See, e.g., U.S. Pat. No. 6,012,902. An electroosmotic pump typically comprises two channels. The pump utilizes electroosmotic pumping of fluid in one channel or region to generate pressure based fluid flow in a connected channel, where the connected channel has substantially no electroosmotic flow generated. For example, an electrokinetic controller applies a voltage gradient to one channel to produce electroosmotically induced pressure within that channel. That pressure is transmitted to a second channel whereupon pressure based flow is achieved. In the present invention, an electroosmotic pump is optionally used to produce pressure-based flow, e.g., in a binding channel. The channel surfaces of the pumping channel typically have charged functional groups associated therewith to produce sufficient electroosmotic flow to generate pressure in the channels in which no electroosmotic flow takes place. See, U.S. Pat. No. 6,012,902 for appropriate types of functional groups.

Hydrostatic, wicking, and capillary forces are also optionally used to provide pressure for fluid flow of materials such as protein mixtures, proteins of interest, dyes, detergents, component-binding-moieties, and the like. See, e.g., U.S. Pat. No. 6,416,642. In these methods, an absorbent material or branched capillary structure is placed in fluidic contact with a region where pressure is applied, thereby causing fluid to move towards the absorbent material or branched capillary structure. The capillary forces are optionally used in conjunction with the electrokinetic or pressure-based flow in the present invention. The capillary action pulls material through a channel. For example a wick is optionally added to a main channel to aid fluid flow by drawing liquid, e.g., a mixture of components, such as proteins, through the channel.

Mechanisms for reducing adsorption of materials during fluid-based flow are described in U.S. Pat. No. 6,458,259. In brief, adsorption of cells, components, proteins, antibodies, and other materials to channel walls or other microscale components during pressure-based flow is optionally reduced by applying an electric field such as an alternating current to the material during flow.

Mechanisms for focusing cells, enzymes, and other components into the center of microscale flow paths, which are useful in increasing assay throughput by regularizing flow velocity, e.g., in pressure based flow, are described in U.S. Pat. No. 6,506,609. In brief, sample materials are focused into the center of a channel by forcing fluid flow from opposing side channels into a main channel comprising the sample materials, or by other fluid manipulations.

In an alternate embodiment, microfluidic systems are incorporated into centrifuge rotor devices, which are spun in a centrifuge. Fluids and particles travel through the device due to gravitational and centripetal/centrifugal pressure forces. For example, samples are optionally transported through a main channel of a planar device using centrifugal force.

The integrated microfluidic system of the invention optionally includes a very wide variety of storage elements for storing reagents to be assessed. These include well plates, matrices, membranes and the like. The reagents are stored in liquids (e.g., in a well on a microtiter plate), or in lyophilized form (e.g., dried on a membrane or in a porous matrix), and can be transported to an array component, region, or channel of the microfluidic device using conventional robotics, or using an electropipettor or pressure pipettor channel fluidly coupled to a region or channel of the microfluidic system.

Another type of reagent optionally included in or introduced into the above devices is a particle set, made from particle member types. The particle set is used for binding or adsorbing components, e.g., a component of interest such as a specific protein, in a binding region. The particle set is optionally used to remove the component of interest from a fluid stream comprising a mixture of components, e.g., by binding the component of interest to a component binding moiety attached to the particle. The component of interest is then typically released from the particle set and detected.

The particle member types typically comprise one of the following: a polymeric material, a silica material, a ceramic material, a glass material, a magnetic material, a metallic material, an organic material, or the like. For example, the particles optionally comprise polymer or ceramic beads. Preferably, the particle member types or beads comprise PVDF, nitrocellulose, or polyamide, e.g., nylon.

The particle member types are optionally stored in a well or reservoir, such as a particle well fluidly coupled to a binding region, and released into the device or system as needed or contained within the binding region or channel in which they will be used. For example, particles are optionally released from a particle well into binding region 110 as shown in FIG. 1, e.g., and stacked using a particle retention area. The particles may be stored and introduced as described above. Additional information on storage, placement and usage of particle sets in microfluidic devices is found, e.g., in U.S. Ser. No. 09/510,626 filed Feb. 22, 2000, entitled “Manipulation of Microparticles In Microfluidic Systems,” by Mehta et al.

II. Affinity Binding

With reference first to FIGS. 2A-D, in one preferred embodiment of the invention, the component binding moiety is preferably reversibly coated to the inner wall surface of a microchannel using a variety of techniques. For example, in one non-limiting example of a binding assay according to the teachings of the present invention, the wall surface of the binding region 210 of a microchannel is coated with biotin derivatized silane to which streptavidin is bound as represented by the reference numeral 220 in FIGS. 2A-D. The streptavidin is then used to bind a biotinylated nickel chelator (e.g., nitrilotriacetic acid (“NTA”)) that binds nickel ions provided as an aqueous solution of nickel salt (e.g., NiCl₂) to the wall of the binding region of the channel. Alternatively, the NTA can be covalently coupled to Thio-Silane coated onto glass channels. The NTA can also be used to coat other metals other than nickel to the wall of the channel, such as metal cations such as cobalt, e.g., cobalt-based TALON™ sold by BD Biosciences Clontech, Palo Alto, Calif., iron, and the like. The nickel, or other appropriate metal cation, is then ready to capture a 6×His-tagged protein or capture antibody 230 specific to the analyte of interest. For example, the binding moiety can include a capture antibody specific for the analyte of interest which is coupled to a polyhistidine tail, either by chemical coupling or genetic engineering techniques, to mediate binding to the nickel coated glass (or silica or quartz or polymeric) wall surface. The polyhistidine-tagged capture antibody binds reversibly to the nickel chelated by NTA on the wall surface of the microchannel in the binding channel region and can be easily released therefrom by flowing an appropriate elution buffer through the system. As described previously, the capture antibody can include at least one label 225, for example, two or more, e.g., four or more, labels associated therewith to facilitate detection thereof in the detection region of the microchannel. The label is optionally a fluorescent label, a chemiluminescent label, an enzyme label, or a colorimetric label. In other embodiments, an associative dye is used to detect components. Such dyes associate with or attach to specific components, e.g., proteins, and are used for detection as described below. These dyes are optionally added with the component binding moiety to the binding affinity region or they can be added later, e.g., prior to introduction of the sample.

A “label” is any composition detectable by spectroscopic, photochemical, biochemical, immunochemical, electrical, optical or chemical means. Useful labels in the present invention include fluorescent dyes (e.g., fluorescein isothiocyanate, Texas red, rhodamine, and the like), radiolabels (e.g., 3H, 125I, 35S, 14C, 32P, 33P, etc.), enzymes (e.g., horseradish peroxidase, alkaline phosphatase etc.), and colorimetric labels such as gold colored glass or plastic (e.g., polystyrene, polypropylene, latex, etc.) beads. The label is coupled directly or indirectly to a component binding moiety (or analyte of interest) of the assay according to methods well known in the art. As indicated above, a wide variety of labels are used, with the choice of label depending on the sensitivity required, ease of conjugation with the component of interest or the component-binding moiety, stability requirements, available instrumentation and disposal provisions. Non-radioactive labels are often attached by indirect means. Generally, a ligand molecule (e.g., biotin) is covalently bound to the component to be labeled. The ligand then binds to an anti-ligand (e.g., streptavidin) molecule which is either inherently detectable or covalently bound to a signal system, such as a detectable enzyme, a fluorescent compound, or a chemiluminescent compound. A number of ligands and anti-ligands are optionally used. Where a ligand has a natural anti-ligand, for example, biotin, thyroxine, or cortisol, it is used in conjunction with the labeled naturally occurring anti-ligand. Alternatively, any haptogenic or antigenic compound is used in combination with an antibody (see, e.g., Coligan (1991) Current Protocols in Immunology, Wiley/Greene, NY; and Harlow and Lane (1989) Antibodies: A Laboratory Manual, Cold Spring Harbor Press, NY for a general discussion of how to make and use antibodies). The components of the invention are also optionally conjugated directly to signal-generating compounds, e.g., by conjugation with an enzyme or fluorophore. Enzymes of interest as labels will primarily be hydrolases, particularly phosphatases, luciferases, esterases and glycosidases, or oxidoreductases, particularly peroxidases. The enzymes are detected in the detection region 150, within a detection volume monitored by a detection device, such as a photodetector, when the enzymes are provided with, e.g. a chromogenic, fluorogenic or lumogenic substrate for the enzyme label where light absorbance, fluorescence or luminescence, respectively are measured in the detection region 150 by the detection device. The substrate for the label enzyme and other components, e.g. co-substrates, ionic or nonionic detergents, chelators, and other needed buffer components for the detection signal optionally are provided through a detection component channel 180 from a detection component reservoir 182. Fluorescent compounds include fluorescein and its derivatives, rhodamine and its derivatives, dansyl, umbelliferone, etc. Chemiluminescent compounds include, e.g., luciferin and 2,3,-dihydrophthalalzinediones, e.g., luminol, and the like.

For example, an antibody is preferably labeled with an enzyme or other label. The enzyme label is optionally a hydrolase such as alkaline phosphatase that is used with dioxetane substrates to generate chemiluminescence that is screened and which can be scanned by the same detector used for the detection of electrophoretic mobility. Alternatively, a linear or 2-dimensional array CCD detector is used to measure the chemiluminescence. There are also fluorescent substrates for alkaline phosphatase that precipitate in situ when hydrolyzed and thereby deposit fluorescent dye where the enzyme label is present. The fluorescence is optionally detected by a fluorescence detector that is scanned along a detection channel region. Furthermore, as described previously, in one preferred embodiment of the invention, the component binding moiety is bound to a nucleic acid marker having at least one label (e.g., two or more labels) associated therewith to facilitate separation and detection of the analyte/antibody complex in the separation channel region of the microchannel.

The sample (e.g., human serum) containing the analyte of interest 240 and other components 250 is flowed into the binding channel region where the analyte 240, if present in the sample, binds to the wall of the channel via the capture antibody 230 to form a bound complex 260. For some period of time, as the sample flows through the channel, the analyte is affinity concentrated in the channel, as much as 5 to 10×, or more. A short wash step using a buffered solution can be used for the concentrated analyte if necessary to remove interfering substances (e.g., human serum) from the analyte of interest.

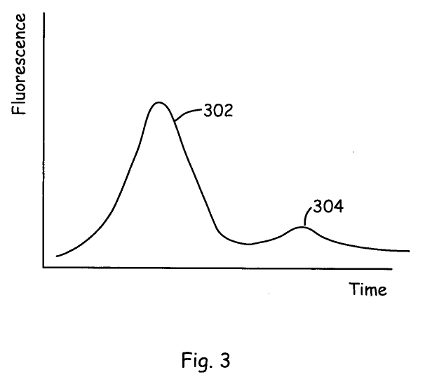

After being bound in the binding channel region by the capture antibody, the analyte/antibody “complex” (e.g., represented by reference numeral 260 in FIG. 2D) can be released from the binding channel region, e.g. eluted therefrom, by flowing an appropriate buffer, e.g., imidazole buffer, through the system. The released analyte/antibody complex 270 can then be separated from any unbound component binding moiety 230, and detected after being released from the binding channel region. Other high salt solutions could optionally be used to perform the elution step such as use of the zwitterions betaine. The use of a zwitterion elution buffer has the advantage of not substantially interfering with the subsequent electrophoretic separation step because of its low electrophoretic conductivity. Release of the complex 260 from the binding channel region can also optionally comprise adjusting, e.g., the temperature or pH in the binding channel region to disfavor the binding of the complex from the derivatized channel wall surface. The temperature is optionally adjusted and/or the pH is changed (i.e., either increased or decreased), e.g., by introducing an acidic or basic reagent into the binding region or by applying heat to the binding channel region (either via external heater means or heater means incorporated into the chip itself, such as resistive metal trace elements coupled to a voltage supply). A representative plot of fluorescence versus time for the assay system of FIGS. 2A-D is shown in FIG. 3 in which peak 302 represents the fluorescent peak for unbound component binding moiety 230, and peak 304 represents the fluorescent peak for the released complex 270.

As described previously, other systems could also be used to reversibly bind the component binding moiety (e.g., capture antibody) to the inner wall surface of the microchannel. For example, the binding moiety can include a disulfide linker such as SPDP (3-(2-Pyridyldithio)propionic Acid N-hydroxy-succinimide ester that can be reduce using dithiothreitol to release the bound analyte/antibody complex. Additionally, reversible ionic interaction could be used as the capture step. For instance, the capture antibody could be coupled to a highly charged molecule, such as DNA, RNA or other charged polymers, that dramatically increases its affinity to the wall surface of the microchannel in the binding channel region or to an ionizable coating, e.g., 3-aminopropylsilane, polylysine, polyarginine, spermidine, cadavarine, or the like, on the channel. A high salt solution, such as a Zwittergen or betaine elution buffer, could then be used to elute the analyte/antibody complex from the channel wall to transfer the complex into the separation channel of the chip for subsequent separation and/or detection thereof.

In addition, in other embodiments, other systems can be used to facilitate separation of the analyte of interest from the component binding moiety itself so that an appropriately labeled analyte of interest can be detected unbound from the analyte/antibody complex formed in the binding channel region. For example, for an avidin-biotin system, modified avidin (e.g., capped avidin) could be used to reduce the affinity of biotin for avidin and thus make the binding of the analyte to the capture antibody reversible. Although binding of biotin to native avidin or streptavidin is essentially irreversible, appropriately modified avidins can bind biotinylated binding moieties reversibly, making them valuable reagents for isolating and purifying biotinylated analytes of interest from complex mixtures (such as human serum). For example, selective nitration of tyrosine residues in the four biotin-binding sites of avidin considerably reduces the affinity of the protein for biotinylated molecules above pH 9. Consequently, biotinylated binding moieties can be adsorbed at neutral pH or below and released at ˜pH 10. Various forms of CaptAvidin agarose available commercially from Molecular Probes (Eugene, Oreg.) are particularly useful for separating and purifying biotin conjugates from complex sample mixtures.

Additionally, modified biotin can be used to provide a means of labeling and separating biomolecules, including live cells, under extremely gentle conditions. For example, DSB-X biotin reagents commercially available from Molecular Probes, which are derivatives of desthiobiotin with an additional seven-atom ‘X’ spacer, have moderate affinity for avidin and streptavidin that is rapidly reversed by low concentrations of free biotin or desthiobiotin at neutral pH and room temperature. This technique permits capture and release of DSB-X biotin-labeled molecules using a DSB-X Biotin Bioconjugate Isolation Kit or Captivate ferrofluid streptavidin also available from Molecular Probes.

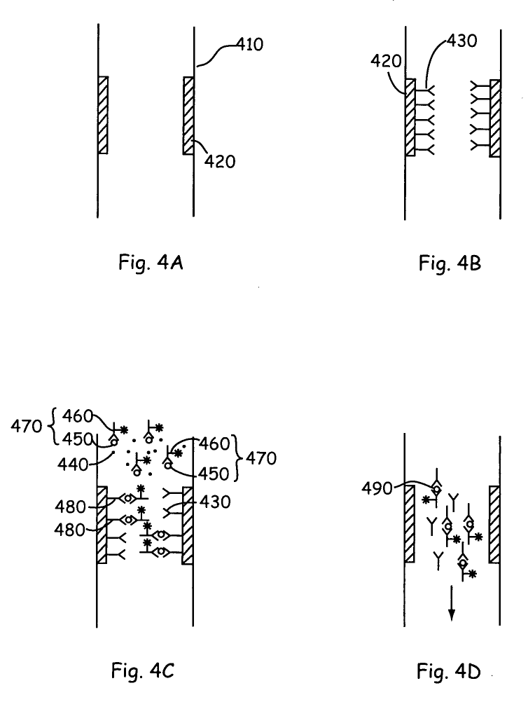

In another embodiment of the present invention useful for performing sensitive binding immunoassays and shown with reference to FIGS. 4A-D, the assay system utilizes at least first and second component binding moieties to perform the assay. A first component binding moiety 430 which is unlabeled and which has an affinity for the analyte of interest is first bound to a coated wall surface 420 in the binding channel region 410, as previously described and as shown in FIG. 4B. The sample 440 in this embodiment, which contains the analyte of interest 450, is preferably pre-incubated with a second component binding moiety 460 which is labeled to form a first labeled component binding moiety/analyte complex 470. The sample containing the first complex 470 is then flowed into the binding channel region where the first labeled complex 470 binds to the first, unlabeled component binding moiety via the analyte of interest to form a second, ternary complex 480 (e.g., capture antibody/analyte/capture antibody) resulting in the analyte (e.g., AFP molecules) being sandwiched between the first component binding moiety 430 and the second component binding moiety 460 (e.g., a fluorescently labeled antibody), as shown in FIG. 4C.

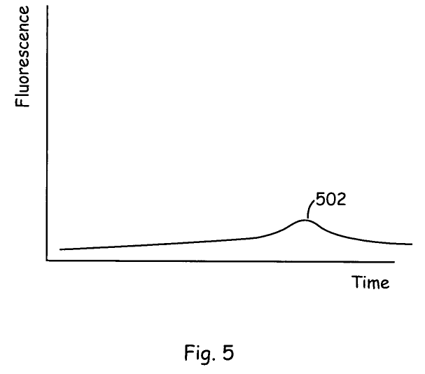

The binding channel region can then be washed to remove any residual test sample, which can be removed through a waste channel as previously described. Release of the ternary complex 480 is then performed as described previously using, for example, an elution buffer such as a imidazole elution buffer, and the released ternary complex 490 is optionally separated, and then detected downstream in the microchannel. A representative plot of fluorescence versus time for the assay system of FIGS. 4A-D is shown in FIG. 5 in which only a single peak 502 is generated which represents the fluorescent peak for the released ternary complex 490. The advantage of this sandwich-type assay technique is that labeled binding moiety is only retained in the binding channel region if analyte is present in the sample to bind to the unlabeled component binding moiety 430. Thus, there will be no fluorescent peak for unbound, labeled component binding moiety as there is for the assay system of FIGS. 2A-D plotted in FIG. 3, which permits easier detection of the analyte complex peak as shown in FIG. 5.

The electrophoretic mobility and detectable signal from the complex of charged, labeled binding moiety and analyte of interest 490 is used to identify and quantitate the analyte of interest. The electrophoretic mobility is used to improve the specificity of the assay by distinguishing analytes with similar binding properties but different charge and size properties. In this case, multiplexed assays can be run for two or more related or unrelated analytes that can be run simultaneously in the same separation channel by designing the charged, labeled binding moiety or mixture of binding moieties to give the different complexes different mobilities. For example, often times the analyte of interest to be detected and quantified has multiple forms or fractions which are of clinical significance to detect separately from one another. For example, in the case of an AFP immunoassay, it is often necessary to distinguish and compare different levels of various fractions of AFP. AFP has been shown to be divided into at least 3 fractions through the lectin-affinity electrophoresis using lens culinaris agglutin (LCA). LCA separates AFP into three bands: LCA-non-reactive (AFP-L1), weakly reactive (AFP-L2); and strongly reactive (AFP-L3). A relative comparison of the levels of AFP L1 to AFP L3, for example, has been shown to be useful as a marker for hepatocellular carcinoma and total AFP as a marker in pregnant women for the potential occurrence of neural tube defects in children. Thus, in the assay system shown in FIGS. 4A-D, for example, the addition of LCA to the sample will allow the different fractions of AFP (e.g., serum levels of AFP L1 and L3) to be separated from one another in the separation region of the microchannel by the application of an electric field to the separation region, and then separately detected downstream in the detection region of the microchannel.

Wash solutions, e.g., BSA solutions, detergent solutions, salt solutions, and the like, of varying stringency are optionally applied to the binding region or to a particle set within the binding region, if used, to remove any unbound components. Stringent wash solutions are optionally applied to remove, e.g., any components bound to the component-binding moiety in a non-specific manner. This works to reduce background levels in later detection of the component of interest. In an alternative embodiment of the invention the component of interest (analyte) may be captured from a supplied sample by the component binding moiety and subsequently released directly, without release of the component binding moiety. For example, the analyte could be a charged molecule of DNA, RNA, or other charged polymer (e.g. a complex of protein and SDS detergent). The component binding moiety, in this example, could be an oppositely-charged coating on the channel, such as 3-aminopropylsilane, polylysine, polyarginine, spermidine, cadavarine, or other such poly-cationic molecule. Following its capture the component of interest may be labeled with a second component-binding moiety which is labeled with a detectable label. For example if the component of interest is DNA, a fluorescently-labeled DNA-binding protein may be used to label the DNA. An optional wash step may be used to remove unwanted interfering species in the sample, the component of interest may be released directly from the component binding moiety by providing dissociating conditions at the site of capture (at the binding region of the microfluidic channel). The dissociating conditions could be any one of the previously mentioned dissociating conditions, e.g. high salt concentration (i.e. high ionic strength), high or low pH (compared to the binding condition). In this embodiment, where the analyte is released independently of the analyte-binding moiety bound to the microchannel wall in the binding channel region of the microfluidic device, the analyte-binding moiety may be retained in the binding channel region of the microfluidic device for use as a component binding moiety for capture of analyte from subsequent samples.

III. Separation of Components

After removing the remaining unwanted sample components from the system through waste channel 120 shown in FIG. 1, and releasing the complex (e.g., complex 260 in FIG. 2D and/or ternary complex 480 in FIG. 4D) into the microchannel, the complex is flowed through the separation channel or region which typically comprises a separation matrix. The separation matrix optionally comprises a polymer, a gel, or a solution, etc. Electrophoretic separation is the separation of substances achieved by applying an electric field to samples in a solution or gel. In its simplest form, it depends on the different velocities with which the substances or components move in the field. The velocities depend, e.g., on the charge and size of the substances.

Preferably, the separation channel region, such as separation channel region 140 in FIG. 1, is a polyacrylamide gel filled channel in which the mixture of complex and unbound, labeled component binding moiety is electrophoretically separated based on charge/mass ratio or molecular weight. If the components are detected as they exit the separation region, e.g., in detection region 150, the components are optionally identified by their retention times.

Other gel electrophoretic media that are optionally placed in a separation channel or region of the invention include silica gels such as Davisil Silica, E. Merck Silica Gel, Sigma-Aldrich Silica Gel (all available from Supelco) in addition to a wide range of silica gels available for various purposes as described in the Aldrich catalogue/handbook (Aldrich Chemical Company, Milwaukee, Wis.). Preferred gel materials include agarose based gels, various forms of acrylamide based gels (reagents available from, e.g., Supelco, SIGMA, Aldrich, Sigma-Aldrich and many other sources), colloidal solutions, such as protein colloids (gelatins) and hydrated starches. For a review of electrophoretic separation techniques and polyacrylamide gels, see, e.g., The Encyclopedia of Molecular Biology, Kendrew (ed.) (1994); and, Gel Electrophoresis of Proteins: A Practical Approach, 2^{nd} edition Hames and Rickwood (eds.) IRL Press, Oxford England, (1990).

Other types of separation matrices are also optionally used and discussed in U.S. Pat. No. 6,306,590. Alternate separation matrix media include low pressure chromatography media, such as non-ionic macroreticular and macroporous resins which adsorb and release components based upon hydrophilic or hydrophobic interactions, e.g., Amberchrom and Amberlite resins (available from Supelco), Dowex, and Duolite (all available from Supelco). Other optional media include affinity media for purification and separation, such as acrylic beads, agarose beads, cellulose, sepharose, or the like. In addition, a wide variety of resins and chromatography media are also available, e.g., from Supelco, Sigma, Aldrich, or the like, for example, biotin resins, dye resins, aluminas, carbopacks, etc. For a review of chromatography techniques and media, see, e.g., Affinity Chromatography—A Practical Approach, Dean et al., (eds.) IRL Press, Oxford (1985); and, Chromatographic Methods, 5^{th} Edition, Braithwaite et al., (1996).

IV. Detectors and Integrated Systems

After separating the complex 270 (or complex 490) from unbound, labeled binding moiety 230, the separated components are flowed through a detection channel region, e.g., detection region 150 in FIG. 1. A detector positioned proximal to a detection region is used to detect the components as they flow through the region, e.g., past a detection window as described above. Likewise, the released complex 270 (or 490 in FIG. 4D) is flowed past the detection region and detected. Detection typically occurs via, e.g., a label moiety attached to the binding moiety. A graphic display of the results is optionally in the form of linear intensity plots or virtual stained gel images.

Detectors for detecting the labeled components of the invention are well known to those of skill in the art. For example, where the label is a radioactive label, a scintillation counter or autoradiography is optionally used. Where the label is a fluorescent label moiety, it is detected by exciting the fluorochrome with the appropriate wavelength of light and detecting the resulting fluorescence. Other label moieties, include, but are not limited to, luminescent labels, color moieties, associative dyes, enzymes, and the like. Various detectors for use in the above methods are discussed in more detail below in the context of an integrated system.

The devices herein optionally include signal detectors, e.g., which detect fluorescence, phosphorescence, radioactivity, pH, charge, absorbance, luminescence, temperature, magnetism, color, or the like. Fluorescent and chemiluminescent detection are especially preferred. Example detectors include photo multiplier tubes, a CCD array, a scanning detector, a galvo-scanner or the like. Proteins, antibodies, nucleic acids, carbohydrates, or other components which emit a detectable signal are optionally flowed past the detector, or, alternatively, the detector can move relative to a channel region to determine protein position (or, the detector can simultaneously monitor a number of spatial positions corresponding to channel regions, e.g., as in a CCD array). For example, for particle member types that are stacked in a detection region, a detector can move relative to the stacked particles and detect them according to position within the stack.

The detector can include, or be operably linked to, a computer, e.g., which has software for converting detector signal information into assay result information, e.g., molecular weight based on retention time or elution time, identity of a protein, or the like.

Signals from arrays are optionally calibrated, e.g., by calibrating the microfluidic system by monitoring a signal from a known or calibrated source. A microfluidic system can also employ multiple different detection systems for monitoring the output of the system. Detection systems of the present invention are used to detect and monitor the materials in a particular channel region (or other detection region). Once detected, the flow rate and velocity of the materials in the channels are also optionally measured and controlled.

Examples of detection systems include optical sensors, temperature sensors, pressure sensors, pH sensors, conductivity sensors, and the like. Each of these types of sensors is readily incorporated into the microfluidic systems described herein. In these systems, such detectors are placed either within or adjacent to the microfluidic device or one or more channels, chambers or conduits of the device, such that the detector is within sensory communication with the device, channel, or chamber. The phrase “proximal,” to a particular element or region, as used herein, generally refers to the placement of the detector in a position such that the detector is capable of detecting the property of the microfluidic device, a portion of the microfluidic device, or the contents of a portion of the microfluidic device, for which that detector was intended. For example, a pH sensor placed in sensory communication with a microscale channel is capable of determining the pH of a fluid disposed in that channel. Similarly, a temperature sensor placed in sensory communication with the body of a microfluidic device is capable of determining the temperature of the device itself. In addition to being used to detect components, e.g., downstream of a separation channel region, such sensors are optionally used to monitor a binding region, e.g., for desirable release conditions. For example, a pH sensor is optionally used to monitor the pH in a binding region to determine when a particular buffer or reagent, e.g., an acidic reagent, or how much of such a reagent should be introduced into the binding channel region to, e.g., release a bound component of interest.

Particularly preferred detection systems include optical detection systems for detecting an optical property of a material within the channels and/or chambers of the microfluidic devices that are incorporated into the microfluidic systems described herein. Such optical detection systems are typically placed adjacent to a microscale channel of a microfluidic device, and are in sensory communication with the channel via an optical detection window that is disposed across the channel or chamber of the device. Optical detection systems include systems that are capable of measuring the light emitted from material within the channel, the transmissivity or absorbance of the material, as well as the material's spectral characteristics. In preferred aspects, the detector measures an amount of light emitted from the material, such as a fluorescent or chemiluminescent material. As such, the detection system will typically include collection optics for gathering a light based signal transmitted through the detection window, and transmitting that signal to an appropriate light detector. Microscope objectives of varying power, field diameter, and focal length are readily utilized as at least a portion of this optical train. The light detectors are optionally photodiodes, avalanche photodiodes, photomultiplier tubes, diode arrays, or in some cases, imaging systems, such as charged coupled devices (CCDs) and the like. In preferred aspects, photodiodes are utilized, at least in part, as the light detectors. The detection system is typically coupled to a computer (described in greater detail below), via an analog to digital or digital to analog converter, for transmitting detected light data to the computer for analysis, storage and data manipulation.

In the case of fluorescent materials such as labeled cells, the detector typically includes a light source that produces light at an appropriate wavelength for activating the fluorescent material, as well as optics for directing the light source through the detection window to the material contained in the channel or chamber. The light source can be any number of light sources that provides an appropriate wavelength, including lasers, laser diodes and LEDs. Other light sources are required for other detection systems. For example, broad band light sources are typically used in light scattering/transmissivity detection schemes, and the like. Typically, light selection parameters are well known to those of skill in the art.

The detector can exist as a separate unit, but is preferably integrated with the controller system, into a single instrument. Integration of these functions into a single unit facilitates connection of these instruments with the computer (described below), by permitting the use of few or a single communication port(s) for transmitting information between the controller, the detector and the computer.

As noted above, either or both of the fluid direction system and/or the detection system are coupled to an appropriately programmed processor or computer which functions to instruct the operation of these instruments in accordance with preprogrammed or user input instructions, receive data and information from these instruments, and interpret, manipulate and report this information to a user. As such, the computer is typically appropriately coupled to one or both of these instruments (e.g., including an analog to digital or digital to analog converter as needed).

The computer typically includes appropriate software for receiving user instructions, either in the form of user input into set parameter fields, e.g., in a GUI, or in the form of preprogrammed instructions, e.g., preprogrammed for a variety of different specific operations. The software then converts these instructions to appropriate language for instructing the operation of the fluid direction and transport controller to carry out the desired operation. For example, the software or control system optionally directs the fluid direction system to transport a fluid-borne sample through a binding channel region and then through a separation region. The computer then receives the data from the one or more sensors/detectors included within the system, e.g., after detection of one or more components. The computer is used to interpret the data, either providing it in a user understood format, or using that data to initiate further controller instructions, in accordance with the programming, e.g., such as in monitoring and control of flow rates, temperatures, pH, applied voltages, and the like.

In the present invention, the computer typically includes software for the monitoring of materials in the channels. Additionally the software is optionally used to control the flow of materials through the channels. For example, monitoring of materials includes monitoring pH or buffer concentration. A control system or software is used to instruct the fluid direction system to introduce more or less buffer to achieve a desired pH or elution concentration in a particular channel region. In addition, the computer optionally includes software for deconvolution of the signal or signals from the detection system. For example, the affinity bound analyte complex can be quantitated by integrating the signal from the detector for the labeled complex and comparing the signal with known calibrators.

Generally, the microfluidic devices described herein are optionally packaged to include reagents for performing the device's preferred function. For example, the kits optionally include any microfluidic device described along with assay components, reagents, sample materials, proteins, antibodies, elution buffers, wash solutions, dyes, particle sets, control materials, or the like. Such kits also typically include appropriate instructions for using the devices and reagents, and in cases where reagents are not predisposed in the devices themselves, with appropriate instructions for introducing the reagents into the channels and/or chambers of the device. In this latter case, these kits optionally include special ancillary devices for introducing materials into the microfluidic systems, e.g., appropriately configured syringes/pumps, or the like (in one preferred embodiment, the device itself comprises a pipettor element, such as an electropipettor for introducing material into channels and chambers within the device). In the former case, such kits typically include a microfluidic device with necessary reagents predisposed in the channels/chambers of the device. Generally, such reagents are provided in a stabilized form, so as to prevent degradation or other loss during prolonged storage, e.g., from leakage. A number of stabilizing processes are widely used for reagents that are to be stored, such as the inclusion of chemical stabilizers (e.g., enzymatic inhibitors, microbicides/bacteriostats, anticoagulants), the physical stabilization of the material, e.g., through immobilization on a solid support, entrapment in a matrix (e.g., a gel), lyophilization, or the like. Kits also optionally include packaging materials or containers for holding microfluidic device, system or reagent elements.

The discussion above is generally applicable to the aspects and embodiments of the invention described in the claims. Moreover, modifications can be made to the method and apparatus described herein without departing from the spirit and scope of the invention as claimed, and the invention can be put to a number of different uses.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above may be used in various combinations. All publications, patents, patent applications, or other documents cited in this application are incorporated by reference in their entirety for all purposes to the same extent as if each individual publication, patent, patent application, or other document were individually indicated to be incorporated by reference for all purposes.

What is claimed is:

1. A method for detecting one or more component of interest in a fluid-borne sample in a microchannel of a microfluidic device, the method comprising the steps of:
(i) flowing the fluid-borne sample comprising the one or more component of interest bound to a first labeled component binding moiety to form a first labeled complex through a binding channel region of the microchannel, which binding channel region comprises at least a second component-binding moiety that is reversibly bound to a wall surface of the binding channel region, thereby binding at least a portion of the one or more components of interest to the second component-binding moiety to form a second labeled complex;
(iii) releasing the second labeled complex comprising the first and second component binding moieties and the one or more component of interest from the binding channel region, thereby releasing the second labeled complex into the microchannel; and,
(iv) flowing the released second labeled complex through a detection channel region of the microchannel and detecting the second labeled complex.

2. The method of claim 1, wherein the protein comprises alpha-fetoprotein.

3. The method of claim 2, wherein the first and second component-binding moieties comprise antibodies specific to the alpha-fetoprotein.

4. The method of claim 3, wherein one of the antibodies comprises a nucleic acid chain bound thereto which includes at least one fluorescent label associated therewith.

5. The method of claim 4, wherein the nucleic acid chain bound to the antibody comprises two or more fluorescent labels.

6. The method of claim 1, wherein the wall surface of the binding channel region of the microchannel is at least partially coated with biotin derivatized silane to which streptavidin is bound, wherein the streptavidin is used to bind a biotinylated nickel chelator that binds nickel to the wall surface of the binding channel region of the microchannel.

7. The method of claim 6, wherein the first component binding moiety is coupled to a polyhistidine tail to mediate binding to the nickel coated wall surface of the binding channel region of the microchannel.

8. The method of claim 7, wherein the step of releasing the second complex from the binding channel region comprises flowing an elution buffer through the microchannel.

9. The method of claim 8, wherein the elution buffer comprises imidazole buffer.

10. The method of claim 1, wherein the first component-binding moiety comprises at least one detectable label moiety.

11. The method of claim 10, wherein the first component binding moiety further comprises a charge modifying moiety.

12. The method of claim 11, wherein the charge modifying moiety comprises a nucleic acid chain.

13. The method of claim 12, wherein the nucleic acid chain is bound to the at least one label moiety.

14. The method of claim 1, wherein the binding channel region comprises a derivatized channel.

15. The method of claim 1, wherein the binding channel region comprises one or more particle set, the particle set comprising a plurality of particle member types.

16. The method of claim 1, wherein flowing the fluid-borne sample through the binding channel region comprises applying pressure to the fluid in the microchannel.

17. The method of claim 1, wherein flowing the fluid-borne sample through the binding channel region comprises electrokinetically flowing the fluid therein.

18. The method of claim 1, wherein releasing the second complex from the binding channel region comprises adjusting the temperature or pH in the binding channel region or introducing one or more releasing reagents into the binding channel region.

19. The method of claim 1, wherein detecting the released second complex comprises optically detecting a fluorescent label moiety fixed to one or more of the component of interest or the first component binding moiety.

20. The method of claim 1, further comprising labeling the first component binding moiety with a fluorescent label and detecting the released second complex by detecting the fluorescent label.

21. The method of claim 1, wherein the second component binding moiety is unlabeled.
